SEQUENCE LISTING (59 sequences)

SEQ ID NO: 1 - DNA, 2545 bp, Amycolatopsis orientalis
gatctctacctcgcgctggcgaacgacacggactgactagccccgcggcgggttgaagatcatcgattccgggttgatctgcttggccttctccatcaggccgtactgcgtcatcaggtcgatcacccgctgcatgcgcaccgggctcatcgcggtcggccacgtgccgaggcgcatcaggctgacggtgtccttgtccactttggcgtagctggtcacggtctgctcgaccaggctgcggttggccgcgtcccgctgtcccttgacgatcgcccgctggaacgccgccgtggtcttcgggttctcctgtgcgtacttggcgctggtcgcccacaccgcgatcggcacgtccaaagtggccccggtcgccgcgtccagcaccggcagcatcccggccttgcgctgcgcctgggtgatgtacggctccaccatgaacgcggcgtcgacgttcttgcgctcgatggccgcctgcatgtccgggaacgggatctcggtgaaggtcaccgtcttgatgtccacaccgttggcctcgagcgcggaccgtgcggtcagttcgacgatgttcgccttggtgttgatcgcgatcttcttgccggccaggtcggctggcttggtgatcgcgttgtccttgccggtcaggatcaggaacatgccctgagcggcctggtaggcgtccgcgaccagcttgatgtccagcacgttcttgtactgcgcggtgaagaacgagacgtagttgccgaatgcgaactgcagttcgccgttggcaaggccgggcaccgccgccgcgccgcccggcagcgacttcagttcgacatcaaggccttcctgggtgaagtagcctttctgctgtgcgatggccagcggtacggtgtccacaatgggcaacgtgccgacgaccactttggtctgctccaaaccgcctgtctggttgggcttttcggtatccccacccagtgccgaacagctggcggcggcgagggcgaggacgcaggacagggctatgcgccacggacgggcgagtgacatgcggggttctcctggcaggcaagacacgatgatctgggccggatcagaccacatcgtccctgttcaacgccagtcgacggaccctactttcgactgaaatatgccagagctcactcgttaagtggcacgaatgtgctatgcatcccatgcaacgggcagcgcccaaaccccgtacgccgcggacttctcgcgcagcttgatctcctcggccggcaccgcgagccgcagcgacgggaacctcgcgaacagccgggtgaagccgatgcgcatctccactctggccagttgctggccgaggcattggtgtataccaccgccgaacgcggcgtgcttgcgtgcgtccactctgtccagttgaaggatatcgggttcgtcgaacaccttcgggtccctattgaccgcgggcagtccgatcgcgacagtgtcgcccttcctgatcatctggccttcgagctccacgtcctccagcgccgcccggttgggcgttccaaggtggacgatcgagaggtagcgcagcagttcctccaccgcgtccgggctgtccagggcagcgatctgctccggatgctgaaggagcgcgaaagtgcctaatcccaacatgttcgcggtggtctcgtgcccggcgacgagcaaaagcaacgcgatgttcgtcagctcttcatcggtcagatcggtgtccgtgatcaagctgccaagcaggtcgtccttggggctcaaccgcttcgtggcgaccagttcagcgatgtagcgggtgagtttgccaagcgccgtcgtcacctcatcctgtgtcttgtccacactggccatgatcgtggtctgctcttggaagaacgcgtgatcggcatacgagacgcccagcagctcgcagatcaccagcgaaggcactggcaacgcgaacgcctgcaccagatcgaccggcggtcctgctttggccatcgcgtcgaggtggtcctcggtgatctggacgatccgcggttcgagttccttgatccgtcgcacggtgaactggctgatcagcatccggcggtaacgcgtgtgctcgggtgcgtccatgttgatgaaccagccgggcgccggggctttcgtggctccgcctggtcgtgggatgacgctgaacaccgggtgcttgtgctcggggcggttgctgaagcgcggatcgatcatgacagtccgcgctgcggcatggctggtcaccagccagccgatgtggccgtcagggaatcgcatcgggctcactggcggaagtttcaccaggtcaggcggtgggtcgaaggggtagcccacggctcggccggtcggtagtgtcactggctcgttcatattttcggagtctactctcatttgatggtggactgtcaaagaagagagttctccggtgtgcagttatcctgctgcggtggatcaaccagcgactgggttgcgggaacgcaagaaggccagaacgaagaccgccatccagcagcacgcgctgcggctgttcaaggagcacggctaccaggccaccacggtcgagcagatc

SEQ ID NO: 2 - DNA, 1206 bp, Amycolatopsis orientalis
  CDS (1)..(1206)
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggccagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgcttttgctcgtcgccgggcacgagaccaccgcgaacatgttgggattaggcactttcgcgctccttcagcatccggagcagatcgctgccctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 3 - protein, 401 aa, Amycolatopsis orientalis
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMASVDKTQDEVTTALGKLTRYIAELVATKRLSPKDDLLGSLITDTDLTDEELTNIALLLLVAGHETTANMLGLGTFALLQHPEQIAALDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 4 - DNA, 2199 bp, Artificial Sequence
  synthetic polynucleotide
atgcgtgtcgactccgaaaacatgaacgagcctgtgaccctccccaccggccgtgccgtgggctaccccttcgaccctcctcctgacctggtgaagcttcctcccgtgagccccatgcgcttccctgacggccacatcggctggctggtgaccagccacgccgctgcgcgtactgtcatgatcgatccccgcttcagcaaccgccccgagcacaagcaccctgtgttcagcgtcatcccccgccccggcggagccactaaggcccccgcgcccggctggttcatcaacatggacgcccccgagcacacccgttaccgccgcatgctgatcagccagttcaccgtgcgccgtatcaaggaactcgaacctcgtatcgtccagatcaccgaggaccacctcgacgcgatggccaaggctggacctcctgtcgatctggtgcaggcgttcgcgttgcctgtgccttcgctggtgatctgcgagctgctgggcgtctcgtacgccgatcacgcgttcttccaggagcagaccaccatcatggcctccgtggacaagactcaggatgaggtgaccaccgcgcttggcaagctcacccgctacatcgctgaactggtcgccactaagcgtttgagccccaaggacgacctgcttggcagcttgatcactgacaccgatctgaccgatgaagagctgaccaacatcgcgttgcttttgctcgtcgccggtcacgagaccaccgcgaacatgttgggactcggcactttcgcgctccttcagcaccccgagcagatcgctgccctggacagccccgacgcggtggaggaactgctgcgctacctctcgatcgtccaccttggaacccccaaccgtgcggcgctggaggacgtggagctcgaaggccagatgatccgcaagggcgacactgtcgcgatcggactgcccgcggtcaaccgtgaccccaaggtgttcgacgaacccgatatccttcagctggaccgtgtggacgctcgcaagcacgccgcgttcggcggtggtattcaccagtgcctcggccagcagctggcccgtgtggagatgcgcatcggcttcacccgtctgttcgcgcgcttcccctcgctgcgtctcgcggtgcccgccgaggagatcaagctgcgcgagaagtccgcggcgtacggtgtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 5 - DNA, 2199 bp, Artificial Sequence; identical to SEQ ID NO: 4
  synthetic polynucleotide

SEQ ID NO: 6 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMASVDKTQDEVTTALGKLTRYIAELVATKRLSPKDDLLGSLITDTDLTDEELTNIALLLLVAGHETTANMLGLGTFALLQHPEQIAALDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 7 - DNA, 33 bp, Artificial Sequence
  synthetic primer
gggggtaccatggccgagatgacagagaaagcc

SEQ ID NO: 8 - DNA, 30 bp, Artificial Sequence
  synthetic primer
gggggtacctcaccaggtgaccgggagttc

SEQ ID NO: 9 - DNA, 40 bp, Artificial Sequence
  synthetic primer
gggggtaccatgagagtagactccgaaaatatgaacgagc

SEQ ID NO: 10 - DNA, 30 bp, Artificial Sequence
  synthetic primer
gggggtaccctatgcatcccatgcaacggg

SEQ ID NO: 11 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgctccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgttaagtgtggacaagacacaggatgaggtgacgacagcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgctgggattaggcactttcgcgctccttcagcatccggagcagatcgctgtcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 12 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggaccgtcatgatcgatccgcgcttcagcaaccgccccgagcacaagcacccggtgttcagcgtcatcccacgaccaggcggagccacgaaagccccggcgcccggctggttctttaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgcttttgctcgtcgccgggcacgagaccaccgcgaacatgttgggattaggcactttcgcgctccttcagcatccggagcagatcgctgccctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 13 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcaccaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgtgctccttcagcacccggagcagatcgctcttctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 14 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgatttcctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcgccaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgctgatcacgcgttcttccaagaacagaccacgatcatgtttagtgtggacaagacacaggatgaggtgacgacggcgcttggcaaactcacccgctacatcgctgaactggtcgccacgaagcggttgagccctaaggacgacctgcttggcagcttgatcacggacaccgatctgaccgatgaagagctgacgaacaccgcgttgcttttgctcgtcgccgggcacgagaccaccgcgaacatgttgggattaggcactttcgcgctccttcagcatccggagcagatcgctgccctggacagcccggacgcggtggaggaactgctgcgctacctctcgatcgtccaccttggagcgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 15 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcaccaacatggacgcacccgagcacacgcgttaccgccggatgctgatcagccagttcaccgtgcgacggatcaaggaactcgaaccgcggatcgtccggatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctaatctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 16 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgctgatcacgcgttcttccaagagcagaccacgatcatgctgagtgtggacaagacacaggataaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctgtcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 17 - DNA, 1206 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccgcctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgcttagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctgtcctggacagcccggacgcggtggaggaactgctgcgctacctctcgatcgtccaccttggaacgcccaaccgggcggcgctggaggacatggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcctcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 18 - DNA, 1206 bp, Artificial Sequence
  synthetic polynucleotide
atgagagtagactccgaaaatatgaacgagccagtgacactaccgaccggccgagccgtgggctaccccctcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgttgagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgcttgcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcatgggatgcatag

SEQ ID NO: 19 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRSEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 20 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFFNMDAPEHTRYRRMLISQFTVRRIKELEPRIVQITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALLLLVAGHETTANMLGLGTFALLQHPEQIAALDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 21 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFTNMDAPEHTRYRRMLISQFTVRRIKELEPRIVQITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALILLVAGHETTANMLGLGTFVLLQHPEQIALLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 22 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFANMDAPEHTRYRRMLISQFTVRRIKELEPRIVQITEDHLDAMAKAGPPVDLVQAFALPVPSLVICELLGVSYADHAFFQEQTTIMFSVDKTQDEVTTALGKLTRYIAELVATKRLSPKDDLLGSLITDTDLTDEELTNTALLLLVAGHETTANMLGLGTFALLQHPEQIAALDSPDAVEELLRYLSIVHLGAPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 23 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFTNMDAPEHTRYRRMLISQFTVRRIKELEPRIVRITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALILLVAGHETTANMLGLGTFALLQHPEQIANLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 24 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDKVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 25 - protein, 401 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDRLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDMELEGQMIRKGDTVAIGLPAVNRDPKVFDEPDILQLDRVDARKHAAFGGGIHQCLGQQLARVEMRIGLTRLFARFPSLRLAVPAEEIKLREKSAAYGVWALPVAWDA

SEQ ID NO: 26 - protein, 401 aa, Artificial Sequence
  synthetic polypeptide
MRVDSENMNEPVTLPTGRAVGYPLDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIACLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDA

SEQ ID NO: 27 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgctccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgttaagtgtggacaagacacaggatgaggtgacgacagcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgctgggattaggcactttcgcgctccttcagcatccggagcagatcgctgtcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 28 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggaccgtcatgatcgatccgcgcttcagcaaccgccccgagcacaagcacccggtgttcagcgtcatcccacgaccaggcggagccacgaaagccccggcgcccggctggttctttaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgcttttgctcgtcgccgggcacgagaccaccgcgaacatgttgggattaggcactttcgcgctccttcagcatccggagcagatcgctgccctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 29 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcaccaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgtgctccttcagcacccggagcagatcgctcttctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 30 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgatttcctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcgccaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgctgatcacgcgttcttccaagaacagaccacgatcatgtttagtgtggacaagacacaggatgaggtgacgacggcgcttggcaaactcacccgctacatcgctgaactggtcgccacgaagcggttgagccctaaggacgacctgcttggcagcttgatcacggacaccgatctgaccgatgaagagctgacgaacaccgcgttgcttttgctcgtcgccgggcacgagaccaccgcgaacatgttgggattaggcactttcgcgctccttcagcatccggagcagatcgctgccctggacagcccggacgcggtggaggaactgctgcgctacctctcgatcgtccaccttggagcgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 31 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcaccaacatggacgcacccgagcacacgcgttaccgccggatgctgatcagccagttcaccgtgcgacggatcaaggaactcgaaccgcggatcgtccggatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatggtcagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctaatctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 32 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgctgatcacgcgttcttccaagagcagaccacgatcatgctgagtgtggacaagacacaggataaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctgtcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 33 - DNA, 2199 bp, Artificial Sequence
  synthetic polynucleotide
atgagagtagactccgaaaatatgaacgagccagtgacactaccgaccggccgagccgtgggctaccccttcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccgcctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgcttagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgctgtcctggacagcccggacgcggtggaggaactgctgcgctacctctcgatcgtccaccttggaacgcccaaccgggcggcgctggaggacatggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcctcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 34 - DNA, 2199 bp, Artificial Sequence
  synthetic polynucleotide
atgagagtagactccgaaaatatgaacgagccagtgacactaccgaccggccgagccgtgggctaccccctcgacccaccgcctgacctggtgaaacttccgccagtgagcccgatgcgattccctgacggccacatcggctggctggtgaccagccatgccgcagcgcggactgtcatgatcgatccgcgcttcagcaaccgccccgagcacaagcacccggtgttcagcgtcatcccacgaccaggcggagccacgaaagccccggcgcccggctggttcatcaacatggacgcacccgagcacacgcgttaccgccggatgctgatcagccagttcaccgtgcgacggatcaaggaactcgaaccgcggatcgtccagatcaccgaggaccacctcgacgcgatggccaaagcaggaccgccggtcgatctggtgcaggcgttcgcgttgccagtgccttcgctggtgatctgcgagctgctgggcgtctcgtatgccgatcacgcgttcttccaagagcagaccacgatcatgttgagtgtggacaagacacaggatgaggtgacgacggcgcttggcaaactcacccgctacatcgctgaactggtcgccacgaagcggttgagccccaaggacgacctgcttggcagcttgatcacggacaccgatctgaccgatgaagagctgacgaacatcgcgttgattttgctcgtcgccgggcacgagaccaccgcgaacatgttgggattaggcactttcgcgctccttcagcatccggagcagatcgcttgcctggacagcccggacgcggtggaggaactgctgcgctacctctcgatcgtccaccttggaacgcccaaccgggcggcgctggaggacgtggagctcgaaggccagatgatcaggaagggcgacactgtcgcgatcggactgcccgcggtcaatagggacccgaaggtgttcgacgaacccgatatccttcaactggacagagtggacgcacgcaagcacgccgcgttcggcggtggtatacaccaatgcctcggccagcaactggccagagtggagatgcgcatcggcttcacccggctgttcgcgaggttcccgtcgctgcggctcgcggtgccggccgaggagatcaagctgcgcgagaagtccgcggcgtacggggtttgggcgctgcccgttgcttgggatgcctctagtgtgctgcaccgtcaccagcctgtcaccatcggagaacccgccgcccgtgcggtgtcccgcaccgtcaccgtcgagcgcctggaccgtatcgccgacgacgtgctgcgcctcgtcctgcgcgacgccggcggaaagactctccccacttggactcccggcgcccacatcgacctcgacctcggcgcgctgtcgcgccagtactccctgtgcggcgcgcccgatgcgcctagctacgagattgccgtgcacctggatcccgagagccgcggcggttcgcgctacatccacgaacagctcgaggtgggaagccctctccgtatgcgcggccctcgtaaccacttcgcgctcgaccccggcgccgagcactacgtgttcgtcgccggcggcatcggcatcacccctgtcctggccatggccgaccacgcccgcgcccgtggatggagctacgaactgcactactgcggccgtaaccgttccggcatggcctacctcgagcgtgtcgccggtcacggtgaccgtgccgccctgcacgtgtccgaggaaggcacccgtatcgacctcgccgccctcctcgccgagcccgcccccggcgtccagatctacgcgtgcggtgccggtcgtctgctcgccggactcgaggacgcgagccgtaactggcccgacggtgcgctgcacgtcgagcacttcacctcgtccctcgcggcgctcgatcctgacgtcgagcacgccttcgacctcgaactgcgtgactcgggtctgaccgtgcgtgtcgaacccacccagaccgtcctcgacgcgttgcgcgccaacaacatcgacgtgcccagcgactgcgaggaaggcctctgcggctcgtgcgaggtcgccgtcctcgacggcgaggtcgaccaccgcgacactgtgctgaccaaggccgagcgtgcggcgaaccgtcagatgatgacctgctgctcgcgtgcctgcggcgaccgtctggccctgcgtctctaa

SEQ ID NO: 35 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRSEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 36 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFFNMDAPEHTRYRRMLISQFTVRRIKELEPRIVQITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALLLLVAGHETTANMLGLGTFALLQHPEQIAALDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 37 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFTNMDAPEHTRYRRMLISQFTVRRIKELEPRIVQITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALILLVAGHETTANMLGLGTFVLLQHPEQIALLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 38 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFANMDAPEHTRYRRMLISQFTVRRIKELEPRIVQITEDHLDAMAKAGPPVDLVQAFALPVPSLVICELLGVSYADHAFFQEQTTIMFSVDKTQDEVTTALGKLTRYIAELVATKRLSPKDDLLGSLITDTDLTDEELTNTALLLLVAGHETTANMLGLGTFALLQHPEQIAALDSPDAVEELLRYLSIVHLGAPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 39 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFTNMDAPEHTRYRRMLISQFTVRRIKELEPRIVRITEDHLDAMAKAGPPVDLVQAFALPVPSLVICELLGVSYADHAFFQEQTTIMVSVDKTQDEVTTALGKLTRYIAELVATKRLSPKDDLLGSLITDTDLTDEELTNIALILLVAGHETTANMLGLGTFALLQHPEQIANLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 40 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDKVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 41 - protein, 732 aa, Artificial Sequence
  synthetic polypeptide
MRVDSENMNEPVTLPTGRAVGYPFDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDRLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIAVLDSPDAVEELLRYLSIVHLGTPNRAALEDMELEGQMIRKGDTVAIGLPAVNRDPKVFDEPDILQLDRVDARKHAAFGGGIHQCLGQQLARVEMRIGLTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 42 - protein, 732 aa, Artificial Sequence
  synthetic polypeptide
MRVDSENMNEPVTLPTGRAVGYPLDPPPDLVKLPPVSPMRFPDGHIGWLVTSHAAARTVMIDPRFSNRPEHKHPVFSVIPRPGGATKAPAPGWFINMDAPEHTRYRRMLISQFTVRRIKELEPRIVQITEDHLDAMAKAGPPVDLVQAFALPVPSLVICELLGVSYADHAFFQEQTTIMLSVDKTQDEVTTALGKLTRYIAELVATKRLSPKDDLLGSLITDTDLTDEELTNIALILLVAGHETTANMLGLGTFALLQHPEQIACLDSPDAVEELLRYLSIVHLGTPNRAALEDVELEGQMIRKGDTVAIGLPAVNRDPKVFDEPDILQLDRVDARKHAAFGGGIHQCLGQQLARVEMRIGFTRLFARFPSLRLAVPAEEIKLREKSAAYGVWALPVAWDASSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGAGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 43 - protein, 16 aa, Artificial Sequence
  synthetic peptide
KAPAPGWFINMDAPEH

SEQ ID NO: 44 - protein, 18 aa, Artificial Sequence
  synthetic peptide
LTNIALLLLVAGHETTAN

SEQ ID NO: 45 - protein, 13 aa, Artificial Sequence
  synthetic peptide
RYLSIVHLGTPNR

SEQ ID NO: 46 - protein, 13 aa, Artificial Sequence
  synthetic peptide
EEIKLREKSAAYG

SEQ ID NO: 47 - protein, 12 aa, Artificial Sequence
  synthetic peptide
FQEQTTIMASVD

SEQ ID NO: 48 - protein, 16 aa, Artificial Sequence
  synthetic peptide
KAPAPGWFANMDAPEH

SEQ ID NO: 49 - protein, 16 aa, Artificial Sequence
  synthetic peptide
KAPAPGWFTNMDAPEH

SEQ ID NO: 50 - protein, 16 aa, Artificial Sequence
  synthetic peptide
KAPAPGWFFNMDAPEH

SEQ ID NO: 51 - protein, 18 aa, Artificial Sequence
  synthetic peptide
LTNTALLLLVAGHETTAN

SEQ ID NO: 52 - protein, 18 aa, Artificial Sequence
  synthetic peptide
LTNIALILLVAGHETTAN

SEQ ID NO: 53 - protein, 18 aa, Artificial Sequence
  synthetic peptide
LTNIALPLLVAGHETTAN

SEQ ID NO: 54 - protein, 13 aa, Artificial Sequence
  synthetic peptide
RYLSIVHLGAPNR

SEQ ID NO: 55 - protein, 13 aa, Artificial Sequence
  synthetic peptide
EEIKLREKSTAYG

SEQ ID NO: 56 - protein, 12 aa, Artificial Sequence
  synthetic peptide
FQEQTTIMTSVD

SEQ ID NO: 57 - protein, 12 aa, Artificial Sequence
  synthetic peptide
FQEQTTIMVSVD

SEQ ID NO: 58 - protein, 12 aa, Artificial Sequence
  synthetic peptide
FQEQTTIMLSVD

SEQ ID NO: 59 - protein, 12 aa, Artificial Sequence
  synthetic peptide
FQEQTTIMFSVD

(19) United States
(12) Patent Application Publication
(10) Pub. No.: US 2010/0217032 A1
(43) Pub. Date: Aug. 26, 2010

(54) PROCESS FOR PREPARING PRAVASTATIN

(76) Inventors: Paul Klaassen, Dordrecht (NL); Adrianus Wilhelmus Hermanus Vollebregt, Naaldwijk (NL); Marco Alexander Van Den Berg, Poeldijk (NL); Marcus Hans, Den Haag (NL); Jan Metske Van Der Laan, Breda (NL)

Correspondence Address: NIXON & VANDERHYE, PC, 901 NORTH GLEBE ROAD, 11TH FLOOR, ARLINGTON, VA, 22203, US

(21) Appl. No.: 12/518,713

(22) PCT Filed: Dec. 11, 2007
(86) PCT No.: PCT/EP07/63638

§ 371 Date: Mar. 23, 2010

(30) Foreign Application Priority Data
Dec. 13, 2006 (EP) 06126046.9

(51) Int. Cl. C12P 7/62; C07K 14/195; C07H 21/04; C07C 69/34

(52) U.S. Cl. 560/194; 530/350; 536/23.7; 435/135

(57) ABSTRACT

The present invention provides a polypeptide having an amino acid sequence according to SEQ ID NO 3, SEQ ID NO 6 or SEQ ID NO 43-59. The present invention also provides a polynucleotide comprising a DNA sequence encoding these polypeptides and a method for isolating polynucleotides encoding polypeptides capable of improving the compactin into pravastatin conversion. Furthermore, the present invention provides a method for producing pravastatin and a pharmaceutical composition comprising pravastatin.

### FIELD OF THE INVENTION

The present invention relates to a method for the production of pravastatin.

### BACKGROUND OF THE INVENTION

Statins are known inhibitors of 3-hydroxy-3-methylbutyryl coenzyme A reductase, the rate-limiting enzyme in cholesterol biosynthesis. As such, statins are able to reduce plasma cholesterol levels in various mammalian species, including man, and the compounds are therefore effective in the treatment of hypercholesterolemia. There are several types of statins on the market, amongst which atorvastatin, pravastatin, compactin, lovastatin and simvastatin. While the former is made via chemical synthesis, the latter four are produced either via direct fermentation or via precursor fermentation. These (precursor) fermentations are carried out by fungi of the genera Penicillium, Aspergillus and Monascus.

Pravastatin is produced in two sequential fermentations. First Penicillium citrinum produces compactin, of which the lactone ring is chemically hydrolyzed and the resulting product is subsequently fed to a cultivation of Streptomyces carbophilus which hydroxylates it to pravastatin. In the context of the present invention the term “hydrolyzed compactin” refers to the non-lactone form of compactin, i.e. in which the lactone ring was opened by reaction with water (FIG. 1); likewise, the term ‘hydrolysis of compactin’ refers to opening of the lactone ring. The industrial species and processes for the production of these metabolites are optimized using different methods. Thus, compactin production by Penicillium citrinum was increased from the original 40 mg/l to 5 g/l. For the biocatalytic conversion a Streptomyces mutant strain with resistance to 3 g/l of mevastatin with an 80% conversion yield was obtained by Metkinen (Metkinen News March 2000, Metkinen Oy, Finland; reviewed by Manzoni and Rollini, 2002, Appl. Microbiol. Biotechnol. 58:555-564). Although commercially viable, this process is far from optimal as compactin titers are low as compared to, for example, industrial amino acid or penicillin G production; moreover, the compactin must be diluted to prevent toxic effects for the Streptomyces strains used in the bioconversion (Hosobuchi et al., 1983, J. Antibiot. 36:887-891) and 20% of the compactin fed is not converted by the Streptomyces strains.

The conversion from compactin into pravastatin is catalyzed by a p450 enzyme of Streptomyces carbophilus (see Matsuoka et al., 1989, Eur. J. Biochem. 184:707-713). There is a common problem with Streptomyces bacteria as they grow in filaments, which results in cultivations with high viscosity leading to low oxygen transfer rates and therefore lower fermentation outputs. Optimally, industrially well equipped species like Escherichia coli, a host widely used in large scale biocatalysis would be useful, but this species does neither have p450 enzymes nor p450 redox regenerating systems. So far, the use of species suitable for fermentation and enzyme production, like Escherichia coli, in the conversion of compactin into pravastatin has not been reported.

Another problem is requirement for co-factor regeneration for the p450 enzyme, which is typically realized by a specific pair of proteins that are present in the host-cell. If this system is not optimal the overall conversion will be substantially lower than 100%, as in the compactin example. Various attempts have been made to isolate alternative species, none of which have a 100% conversion rate (see U.S. Pat. No. 6,905,851, U.S. Pat. No. 6,365,382, US 2005/0153422, US 2004/0253692 and US 2004/0209335). Moreover, none of these show a real improvement over Streptomyces carbophilus. Also reported are species with extreme high resistance towards compactin, but with an inefficient conversion (U.S. Pat. No. 6,306,629, U.S. Pat. No. 6,750,366). Others suggest using family shuffling as a method of improving the conversion rate of known p450 enzymes, but do not show any data (U.S. Pat. No. 6,605,430) as indeed this will be very difficult since p450 enzymes can be very substrate specific, do not have much sequence homology and need a set of specific enzymes for co-factor regeneration. It has been tried to solve this latter problem by isolating species that use different enzymes for the conversion. One particular example in this field is an Actinomadura species capable of converting compactin into pravastatin with a maximum conversion rate of 78% (Peng and Demain (1998, J. Ind. Microbiol. Biotechnol. 20:373-375; U.S. Pat. No. 6,274,360)). So despite all efforts, Streptomyces carbophilus, with only 80% conversion rate, is still used as the industrial species of choice for the formation of pravastatin and improvements are highly desirable.

### DESCRIPTION OF THE INVENTION

In the context of the present invention, the term “conservative substitution” is intended to mean that a substitution in which the amino acid residue is replaced with an amino acid residue having a similar side chain. These families are known in the art and include amino acids with basic side chains (e.g. lysine, arginine and histidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine tryptophan, histidine).

The term “isolated polynucleotide or nucleic acid sequence” as used herein refers to a polynucleotide or nucleic acid sequence which is essentially free of other nucleic acid sequences, e.g., at least 20% pure, preferably at least 40% pure, more preferably at least 60% pure, even more preferably at least 80% pure, most preferably at least 90% pure as determined by agarose electrophoresis. For example, an isolated nucleic acid sequence can be obtained by standard cloning procedures used in genetic engineering to relocate the nucleic acid sequence from its natural location to a different site where it will be reproduced.

The term “pravastatin” is defined as the 6′-hydroxyl variant of compactin with an α or β-configuration, or a mixture of both α and β-configurations. It is important to mention here that in the scientific literature, the term pravastatin is solely used for the β-configuration of the 6′-hydroxyl variant of compactin, while the α variant is named epi-pravastatin. However, this invention described a general efficient method to generate 6-hydroxyl variants of compactin. Therefore, the term pravastatin applies for both the α and β forms.

It is an object of the present invention to provide for an effective and industrial applicable method for converting compactin into pravastatin. It is another object of the invention to use a novel p450 enzyme from Amycolatopsis orientalis to convert compactin into pravastatin. The present invention solves the problems encountered in prior art processes, by providing a process in which compactin hydroxylation is performed efficiently in Escherichia coli. Also provided is a process in which compactin hydroxylation is performed with 100% conversion. More specifically a process is provided wherein compactin is contacted with the Amycolatopsis orientalis compactin hydroxylase enzyme (encoded by the cmpH gene), either by contacting whole cells or a cell-free extract of Amycolatopsis orientalis with compactin. Preferably, a process is provided wherein the compactin hydroxylase (cmpH) is obtained from Amycolatopsis orientalis and transferred to another host species. Preferably, this host is resistant to high levels of compactin and capable of compactin production.

In a first aspect, provided is a polypeptide selected from the group consisting of a polypeptide having an amino acid sequence according to SEQ ID NO 3 and a polypeptide having an amino acid that is substantially homologous to the sequence of SEQ ID NO 3, the polypeptide displaying compactin hydroxylase activity.

In a first embodiment, said polypeptide hydroxylates compactin with an efficiency of at least 50%, preferably at least 70%, more preferably at least 80%, still more preferably at least 90%, most preferably 99%. Preferably the product of said hydroxylation is pravastatin.

As part of the present invention it is demonstrated that the industrial application of the currently available compactin hydroxylases is limited to species from the class of Actinomycetes; i.e. they cannot be transferred to species more amenable to industrial scale fermentations like Escherichia coli or filamentous fungi such as Aspergillus or Penicillium species. The compactin hydroxylase genes described by the present invention do not have this problem. The activity of the novel polypeptides encoded by these genes can therefore be characterized as follows: they enable application in other species than Actinomycetes, for example in Escherichia coli, and/or they enable an efficient hydroxylation of compactin to pravastatin with a conversion efficiency of at least 80%. In the context of the invention, an efficiency of at least 80% means that at least 80% of the compactin is converted into pravastatin.

A polypeptide with an amino acid sequence that is substantially homologous to SEQ ID NO 3 is defined as a polypeptide with an amino acid sequence with a degree of identity to the specified amino acid sequence of at least 50%, preferably at least 60%, more preferably at least 75%, still more preferably at least 90%, most preferably at least 95%, still most preferably at least 97%, ultimately at least 98%, still more ultimately at least 99%, the substantially homologous peptide displaying compactin hydroxylase activity. A substantially homologous polypeptide encompasses polymorphisms that may exist in cells from different populations or within a population due to natural allelic or intra-strain variation. A substantially homologous polypeptide may further be derived from a species other than the species where the specified amino acid and/or DNA sequence originates from, or may be encoded by an artificially designed and synthesized DNA sequence. DNA sequences related to the specified DNA sequences and obtained by degeneration of the genetic code are also part of the invention. Homologues also encompass biologically active fragments of the full-length sequence, still displaying compactin hydroxylase activity.

The degree of identity between two amino acid sequences refers to the percentage of amino acids that are identical between the two sequences. The degree of identity is determined using the BLAST algorithm, which is described in Latched et al. (1990, J. Mol. Biol. 215:403-410). BLAST analysis software is available through the National Center for Biotechnology Information (http://www.ncbi.nlm.nih.gov/). The BLAST algorithm parameters W, T, and X determine the sensitivity and speed of the alignment. The BLAST program uses as defaults a word length (W) of 11, the BLOSUM62 scoring matrix (see Henikoff and Henikoff, 1989, Proc Natl. Acad. Sci. USA 89:10915) alignments (B) of 50, expectation (E) of 10, M=5 and N=−4.

Substantially homologous polypeptides may contain only conservative substitutions of one or more amino acids of the specified amino acid sequences or substitutions, insertions or deletions of non-essential amino acids. Accordingly, a non-essential amino acid is a residue that can be altered in one of these sequences without substantially altering the biological function. For example, guidance concerning how to make phenotypically silent amino acid substitutions is provided in Bowie et al. (1990, Science 247:1306-1310) indicating that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. These studies have revealed that proteins are surprisingly tolerant to amino acid substitutions and reveal which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

In a second embodiment, variants leading to an improved catalytic function (i.e. conversion of compactin into pravastatin) may be obtained by modifying the polynucleotide sequences encoding compactin hydroxylase. Among such modifications are:
Improving the codon usage in such a way that the codons are adapted to the host species used for expressing the compactin hydroxylase
Improving the codon pair usage in such a way that the codons are adapted to the host species used for expressing the compactin hydroxylase
Addition of stabilizing sequences to the genomic information encoding compactin hydroxylase resulting in mRNA molecules with an increased half life
Error prone PCR to introduce random mutations, followed by a screening of obtained variants (essentially as described in example 4) and isolating of variants with improved kinetic properties
Family shuffling of related variants of compactin hydroxylase, followed by a screening of obtained variants (essentially as described in example 4) and isolating of variants with improved kinetic properties

Preferred methods to isolate variants with improved kinetic properties are described in WO03010183 and WO0301311.

An improved catalytic function is obtained when an improved polynucleotide is obtained that encodes compactin hydroxylase with improved functionality. As part of the present invention, it has surprisingly been found that the ratio between the β-configuration of the 6-hydroxyl variant of compactin (i.e. the pharmaceutically active pravastatin isomer) and the α-configuration of the 6-hydroxyl variant of compactin could be improved significantly using the improved polypeptide sequences of SEQ ID NO 19, 20, 21, 22, 23, 24, 25 or 26 or sequences substantially homologous thereto.

In addition, it was established that certain stretches within the sequence of the polypeptides of the first aspect of the present invention are directly involved in the catalytic machinery of the hydroxylation of compactin. These are SEQ ID NO 43, 44, 45, 46 and 47. An improved catalytic function could be obtained by introducing modifications in any or in all of SEQ ID NO 43-47. Preferably, any or all of SEQ ID NO 43-47 are modified by replacing a single amino acid, two amino acids, three amino acids or at most four amino acids. It has been established that the following modifications lead to an improved catalytic function. For SEQ ID NO 43 the preferred modifications are SEQ ID NO 48, 49 and 50, for SEQ ID NO 44 the preferred modifications are SEQ ID NO 51, 52 and 53, for SEQ ID NO 45 the preferred modification is SEQ ID NO 54, for SEQ ID NO 46 the preferred modification is SEQ ID NO 55 and for SEQ ID NO 47 the preferred modifications are SEQ ID NO 56, 57, 58 and 59. Stretches suitable for contributing to the hydroxylation of compactin are also SEQ ID NO 43-59 wherein one, two or three amino acids are replaced with alternate amino acids.

In a third embodiment, there is provided a polynucleotide or nucleic acid sequence comprising a DNA sequence encoding the polypeptides mentioned above. This may be an isolated polynucleotide of genomic, cDNA, RNA, semi-synthetic, synthetic origin, or any combinations thereof. In particular, a specific DNA sequence is provided encoding the polypeptide of SEQ ID NO 3, i.e. SEQ ID NO 1 or 2. More preferably, the specific DNA sequence is provided encoding the polypeptides of SEQ ID 19-26, i.e. SEQ ID 11-18. Unless otherwise indicated, all nucleotide sequences determined by sequencing a DNA molecule herein are determined using an automated DNA sequencer and all amino acid sequences of polypeptides encoded by DNA molecules determined herein were predicted by translation of a DNA sequence determined as above. Therefore, for any DNA sequence determined by this automated approach, any nucleotide sequence determined may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. The person skilled in the art is capable of identifying such erroneously identified bases and knows how to correct for such errors.

The polypeptides and the encoding nucleic acid sequences of the first aspect of the invention may be obtained from any prokaryotic cell, preferably from Actinomycetes. Preferred actinomycetes species include, but are not limited to, strains of Streptomyces, Amycolatopsis, Pseudonocardia, Micromonospora, Nocardia and Actinokineospora. In a preferred embodiment, the nucleic acid sequence encoding a polypeptide of the present invention is obtained from a strain of Amycolatopsis orientalis.

DNA sequences of the invention may be identified by hybridization. Nucleic acid molecules corresponding to variants (e.g. natural allelic variants) and homologues of the DNA of the invention can be isolated based on their homology to the nucleic acids disclosed herein using these nucleic acids or a suitable fragment thereof, as a hybridization probe according to standard hybridization techniques, preferably under highly stringent hybridization conditions. Alternatively, one could apply in silico screening through the available genome databases. “Stringency” of hybridization reactions is readily determinable by one of ordinary skill in the art. For additional details and explanation of stringency of hybridization reactions, see Ausubel et al. (1995, Current Protocols in Molecular Biology, Wiley Interscience Publishers).

The nucleic acid sequence may be isolated by e.g. screening a genomic or cDNA library of the microorganism in question. Once a nucleic acid sequence encoding a polypeptide having an activity according to the invention has been detected with e.g. a probe derived from SEQ ID NO 2, the sequence may be isolated or cloned by utilizing techniques which are known to those of ordinary skill in the art (see Sambrook et al., 1989, Molecular Cloning, A Laboratory Manual, 2d edition, Cold Spring Harbor, New York). The cloning of the nucleic acid sequences of the present invention from such (genomic) DNA can also be effected, e.g. by using methods based on polymerase chain reaction (PCR) or antibody screening of expression libraries to detect cloned DNA fragments with shared structural features (See, e.g., Innis et al., 1990, PCR: A Guide to Methods and Application, Academic Press, New York.).

The sequence information as provided herein should not be so narrowly construed as to require inclusion of erroneously identified bases. The specific sequences disclosed herein can be readily used to isolate the complete gene from Actinomycetes, in particular Amycolatopsis orientalis, which in turn can easily be subjected to further sequence analyses thereby identifying sequencing errors.

Unless otherwise indicated, all nucleotide sequences determined by sequencing a DNA molecule herein where determined using an automated DNA sequencer and all amino acid sequences of polypeptides encoded by DNA molecules determined herein were predicted by translation of a DNA sequence determined as above. Therefore, as is known in the art for any DNA sequence determined by this approach, any nucleotide sequence determined herein may contain errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. The person skilled in the art is capable of identifying such erroneously identified bases and knows how to correct for such errors.

In a fourth embodiment the invention provides for an improved compactin hydroxylase enzyme by fusing the polypeptide of SEQ ID NO 3 to a so-called reductase domain to form the polypeptide of SEQ ID NO 6 and displaying compactin hydroxylase activity. The scope of this invention is not limited to this specific amino acid sequence, but includes polypeptides having an amino acid sequence that is “substantially homologous” to the sequence of SEQ ID NO 6, which is defined as a polypeptide having an amino acid sequence possessing a degree of identity to the specified amino acid sequence of at least 60%, preferably at least 70%, more preferably at least 80%, still more preferably at least 85%, still more preferably at least 90%, still more preferably at least 95%, still more preferably at least 98%, most preferably at least 99%, the substantially homologous peptide displaying compactin hydroxylase activity. A substantially homologous polypeptide may encompass polymorphisms that may exist in cells from different populations or within a population due to natural allelic or intra-strain variation. A substantially homologous polypeptide may further be derived from a species other than the species where the specified amino acid and/or DNA sequence originates from, or may be encoded by an artificially designed and synthesized DNA sequence. DNA sequences related to the specified DNA sequences and obtained by degeneration of the genetic code are also part of the invention. Homologues may also encompass biologically active fragments of the full-length sequence, still displaying compactin hydroxylase activity. The person skilled in the art will understand that the hydroxylase part of this fusion protein may be exchanged for non-homologous, but still functional equivalent sequences, like other p450 enzymes capable of hydroxylating compactin, such as the Streptomyces carbophilus p450sca-2 gene, provided that the fused protein displays compactin hydroxylation pravastatin. Also, the reductase domain can be exchanged for non-homologous, but still functionally equivalent sequences, for example ferredoxins and ferredoxin reductases, provided that the fused protein displays compactin hydroxylation towards pravastatin. Alternative reductase domains that can be used are for example the reductase domains of the self-sufficient P450 enzymes from Bacillus megaterium, P450 BM3, NCBI Genbank accession number gi142797. Preferred fused polypeptides are the congeners of the improved polypeptides of SEQ ID NO 19-26, namely SEQ ID NO 35, 36, 37, 38, 39, 40, 41 or 42 or sequences substantially homologous thereto. In addition, also the specific DNA sequences encoding the polypeptides of SEQ ID NO 34-42, i.e. SEQ ID NO 27-34 are part of the present invention. Alternatively, improvements in the catalytic function as described in the second embodiment may also be performed on the reductase region.

In a second aspect, the present invention discloses the use of a polynucleotide of the first aspect in recombinant host strains. More particularly, disclosed is a method for producing pravastatin comprising the steps of:
(i) transforming a host cell of interest with a polynucleotide comprising the gene of interest encoding compactin hydroxylase,
(ii) selecting clones of transformed cells,
(iii) cultivating said selected cells,
(iv) optionally processing said cultivated cells (i.e. immobilizing),
(v) feeding compactin to said cultivated cells,
(vi) isolating pravastatin from said cultivations.

The choice of a host cell in the method of the present invention will to a large extent depend upon the source of the nucleic acid sequence (gene) of interest encoding a polypeptide. Preferably, the host cell is a prokaryotic cell. In a preferred embodiment, the prokaryotic host cell is a cell of a species cited as species from which the polynucleotide of the first or second aspect may be obtained, examples of which are, but are not limited to, Streptomyces species (i.e. Streptomyces carbophilus, Streptomyces flavidovirens, Streptomyces coelicolor, Streptomyces lividans, Streptomyces exfoliatus) or Amycolatopsis species (i.e. Amycolatopsis orientalis). In the most preferred situation, the host cell is a host cell suitable for large scale fermentation, examples of which are, but are not limited to, Streptomyces species (i.e. Streptomyces avermitilis, Streptomyces lividans, Streptomyces clavuligerus) or Bacillus species (i.e. Bacillus subtilus, Bacillus amyloliquefaciens, Bacillus licheniformis) or Corynebacterium species (i.e. Corynebacterium glutamicum) or Escherichia species (i.e. Escherichia coli). Even more preferably, the host cell is a eukaryotic cell, such as Saccharomyces, Aspergillus or Penicillium species, suitable examples of which are the yeast Saccharomyces cerevisiae or the filamentous fungi Aspergillus niger, Penicillium chrysogenum or Penicillium citrinum.

Nucleic acid constructs, e.g. expression constructs, may contain a selection marker gene and the polynucleotide of the invention (compactin hydroxylase), each operably linked to one or more control sequences, which direct the expression of the encoded polypeptide in a suitable expression host. The nucleic acid constructs may be on separate fragments or, preferably, on one DNA fragment. Expression will be understood to include any step involved in the production of the polypeptide and may include transcription, post-transcriptional modification, translation, post-translational modification and secretion. The term “nucleic acid construct” is synonymous with the term “expression vector” or “cassette” when the nucleic acid construct contains all the control sequences required for expression of a coding sequence in a particular host organism. The term “control sequences” is defined herein to include all components, which are necessary or advantageous for the expression of a polypeptide. Each control sequence may be native or foreign to the nucleic acid sequence encoding the polypeptide. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences include a promoter, and transcriptional and translational stop signals. The term “operably linked” is defined herein as a configuration in which a control sequence is appropriately placed at a position relative to the coding sequence of the DNA sequence such that the control sequence directs the production of a polypeptide.

The control sequence may include an appropriate promoter sequence containing transcriptional control sequences. The promoter may be any nucleic acid sequence, which shows transcription regulatory activity in the cell including mutant, truncated, and hybrid promoters, and may be obtained from genes encoding extra cellular or intracellular polypeptides. The promoter may be either homologous or heterologous to the cell or to the polypeptide. Preferred promoters for prokaryotic cells are known in the art and can be, for example, strong promoters ensuring high level messenger RNA. The promotor used in the expression cassette according to the invention may be selected from the well-known set of inducible promoters for highly expressed operons/genes like the lactose operon (lac, lacUV5), the arabinose operon (ara), the tryptophan operon (trp), and the operon encoding enzymes common to the biosynthesis of all aromatic amino acids (aro), or functional hybrids of these, e.g. the tac promoter, which is a fusion of the trp and the lac promoter (Amann et al., 1983, Gene 25:161-178). Alternatively, constitutive promoters can be used providing for a constant supply of messenger RNA throughout the cell's life. Any other useful promoters can be found among others at the NCBI website (http://www.ncbi.nlm.nih.gov/entrez/).

In a preferred embodiment, the promoter may be derived from a gene, which is highly expressed (defined herein as the mRNA concentration with at least 0.5% (w/w) of the total cellular mRNA). In another preferred embodiment, the promoter may be derived from a gene, which is medium expressed (defined herein as the mRNA concentration with at least 0.01% until 0.5% (w/w) of the total cellular mRNA). In another preferred embodiment, the promoter may be derived from a gene, which is low expressed (defined herein as the mRNA concentration lower than 0.01% (w/w) of the total cellular mRNA).

In an even more preferred embodiment, Micro Array data is used to select genes, and thus promoters of those genes, that have a certain transcriptional level and regulation. In this way one can adapt the gene expression cassettes optimally to the conditions it should function in.

Alternatively, one could clone random DNA fragments in front of the polynucleotides of this invention. These can be isolated via a so-called direct selection approach. Using a promoter-less selectable marker gene (i.e. kanamycin resistance) one can clone random DNA fragments in front of this gene and easily screen for active promoters, as these should facilitate growth on media containing kanamycin. These DNA fragments can be derived from many sources, i.e. different species, PCR amplified, synthetically and the like. Subsequently, the sequences can be isolated and cloned in front of the polynucleotides of this invention. Similar strategies can be used to improve translation of the messenger RNA pool by introducing, via recDNA methodology, 5′-untranslated leader regions from efficiently translated messenger RNA's like those obtainable from the tuf gene encoding the highly expressed Elongation Factor Tu protein, or modified or synthetic variants of the tryptophan operon.

The control sequence may also include a suitable transcription terminator sequence, a sequence recognized by a prokaryotic cell to terminate transcription. The terminator sequence is operably linked to the 3′ terminus of the nucleic acid sequence encoding the polypeptide. Any terminator, which is functional in the cell, may be used in the present invention. Preferred terminators for prokaryotic cells are obtained either from the native gene to be expressed or from sources like rRNA genes or viral operons, e.g. the ribosomal RNA terminator, or the fd terminator (Sambrook et al., 1989. Molecular Cloning 2^{nd} edition; CSH Press).

For secretion of a polypeptide, the control sequence may include a signal pep-tide-encoding region, coding for an amino acid sequence linked to the amino terminus of the polypeptide, which can direct the encoded polypeptide into the cell's secretory pathway. The 5′-end of the coding sequence may inherently contain a signal peptide-coding region naturally linked in translation reading frame with the segment of the coding region, encoding the secreted polypeptide. Alternatively, the 5′-end of the coding sequence may contain a signal peptide-coding region, foreign to the coding sequence. The foreign signal peptide-coding region may be required where the coding sequence does not normally contain a signal peptide-coding region. Alternatively, the foreign signal peptide-coding region may simply replace the natural signal peptide-coding region in order to obtain enhanced secretion of the polypeptide.

The nucleic acid construct may be an expression vector. The expression vector may be any vector (e.g. a plasmid or virus), which can be conveniently subjected to recombinant DNA procedures and can bring about the expression of the nucleic acid sequence encoding the polypeptide. The choice of the vector will typically depend on the compatibility of the vector with the cell into which the vector is to be introduced. The vectors may be linear or closed circular plasmids.

In another embodiment, the expression cassette last mentioned is additionally modified by replacement of the original promoter by the trp promoter or the aro promoter. In order to fully exploit the basic improvement of expression efficiency, additional modifications relating to the increased gene expression, messenger RNA translation and plasmid stability may be applied to the recDNA constructs used to create the actual production strain e.g. addition of the Transcription terminator of phage fd, or the introduction of the partitioning function par from plasmid pSC101 (Churchward et al., 1983. Nucl. Acid. Res. 11:5645-5659).

To increase production of the desired protein one can insert the expression cassette on extrachromosomal elements, such as plasmids ColE1, ColD, R1162, RK2 or derivatives that are present in predetermined low copy numbers or, often dynamic, high copy numbers and that are capable of propagation or autonomous replication in e.g. Escherichia coli strains HB101, B7, RV308, DH1, HMS174, W3110, BL21.

The vector may be an autonomously replicating vector, i.e. a vector, which exists as an extrachromosomal entity, the replication of which is independent of chromosomal replication, e.g. a plasmid, an extrachromosomal element, a minichromosome, or an artificial chromosome. Alternatively, the vector may be one which, when introduced into the cell, is integrated into the genome and replicated together with the chromosome(s) into which it has been integrated. The integrative cloning vector may integrate at random or at a predetermined target locus in the chromosomes of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least at least 0.1 kb, even preferably at least 0.2 kb, more preferably at least 0.5 kb, even more preferably at least 1 kb, most preferably at least 2 kb. The vector system may be a single vector or plasmid or two or more vectors or plasmids, which together contain the total DNA to be introduced into the genome of the host cell.

The DNA constructs may be used on an episomal vector. Preferably, the constructs are integrated in the genome of the host strain.

In another embodiment, the application of the polypeptides of the present invention can be improved by deleting one or more of the endogenous genes from the genome of the host strain encoding enzymes limiting the pravastatin yields. Examples of such enzymes are, but are not limited to, enzymes that hydrolyze the side chains of compactin or pravastatin.

In a preferred embodiment the cmpH gene (SEQ ID NO 1), all homologous sequences, the cmpH fusion to a reductase domain (SEQ ID NO 4) and all functional equivalents encoding compactin hydroxylase can be expressed in a compactin producing host cell to produce pravastatin. In case of a prokaryotic host one can apply all aspects of functional expression in such a host as described above. In case of a eukaryotic host cell one preferably adapts the expression constructs towards efficient expression in such hosts. Preferably, the host cell is a fungus, more preferably a filamentous fungus, most preferably, the fungal host cell is a cell which produces statins, preferably compactin. Examples of which are, but are not limited to, Aspergillus species (i.e. Aspergillus terreus), or Penicillium species (i.e. Penicillium citrinum or chrysogenum), or Monascus species (i.e. Monascus ruber or paxii).

Preferred promoters for filamentous fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase IacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, etc or any other, and can be found among others at the NCBI website (http://www.ncbi.nlm.nih.gov/entrez/).

In a preferred embodiment, the promoter may be derived from a gene, which is highly expressed (defined herein as the mRNA concentration with at least 0.5% (w/w) of the total cellular mRNA). In another preferred embodiment, the promoter may be derived from a gene, which is medium expressed (defined herein as the mRNA concentration with at least 0.01% until 0.5% (w/w) of the total cellular mRNA). In another preferred embodiment, the promoter may be derived from a gene, which is low expressed (defined herein as the mRNA concentration lower than 0.01% (w/w) of the total cellular mRNA).

In an even more preferred embodiment, Micro Array data is used to select genes, and thus promoters of those genes, that have a certain transcriptional level and regulation. In this way one can adapt the gene expression cassettes optimally to the conditions it should function in.

The control sequence may also include a suitable transcription terminator sequence, a sequence recognized by a filamentous fungal cell to terminate transcription. The terminator sequence is operably linked to the 3′ terminus of the nucleic acid sequence encoding the polypeptide. Any terminator, which is functional in the cell, may be used in the present invention. Preferred terminators for filamentous fungal cells are obtained from the genes encoding Aspergillus oryzae TAKA amylase, Aspergillus niger glucoamylase, Aspergillus nidulans anthranilate synthase, Aspergillus niger alpha-glucosidase, trpC gene and Fusarium oxysporum trypsin-like protease.

The control sequence may also include a suitable leader sequence, a non-translated region of an mRNA, which is important for translation by the filamentous fungal cell. The leader sequence is operably linked to the 5′-terminus of the nucleic acid sequence encoding the polypeptide. Any leader sequence, which is functional in the cell, may be used in the present invention. Preferred leaders for filamentous fungal cells are obtained from the genes encoding Aspergillus oryzae TAKA amylase and Aspergillus nidulans triose phosphate isomerase and Aspergillus niger glaA.

The control sequence may also include a polyadenylation sequence, operably linked to the 3′-terminus of the nucleic acid sequence and which, when transcribed, is recognized by the filamentous fungal cell as a signal to add polyadenosine residues to transcribed mRNA. Any polyadenylation sequence, functional in the cell, may be used in the present invention. Preferred polyadenylation sequences for filamentous fungal cells are obtained from the genes encoding Aspergillus oryzae TAKA amylase, Aspergillus niger glucoamylase, Aspergillus nidulans anthranilate synthase, Fusarium oxysporum trypsin-like protease and Aspergillus niger α-glucosidase.

The nucleic acid construct may be an expression vector. The expression vector may be any vector (e.g. a plasmid or virus), which can be conveniently subjected to recombinant DNA procedures and can bring about the expression of the nucleic acid sequence encoding the polypeptide. The choice of the vector will typically depend on the compatibility of the vector with the cell into which the vector is to be introduced. The vectors may be linear or closed circular plasmids.

The vector may be an autonomously replicating vector, i.e. a vector, existing as an extrachromosomal entity, the replication of which is independent of chromosomal replication, e.g. a plasmid, an extrachromosomal element, a minichromosome, or an artificial chromosome. An autonomously maintained cloning vector for a filamentous fungus may comprise the AMA1-sequence (see e.g. Aleksenko and Clutterbuck (1997), Fungal Genet. Biol. 21: 373-397). Alternatively, the vector may be one which, when introduced into the cell, is integrated into the genome and replicated together with the chromosome(s) into which it has been integrated. The integrative cloning vector may integrate at random or at a predetermined target locus in the chromosomes of the host cell. Preferably, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least at least 0.1 kb, even preferably at least 0.2 kb, more preferably at least 0.5 kb, even more preferably at least 1 kb, most preferably at least 2 kb. The vector system may be a single vector or plasmid or two or more vectors or plasmids, which together contain the total DNA to be introduced into the genome of the host cell.

The DNA constructs may be used on an episomal vector. Preferably, the constructs are integrated in the genome of the host strain.

Fungal cells are transformed using co-transformation, i.e. along with gene(s) of interest also a selectable marker gene is transformed. This can be either physically linked to the gene of interest (i.e. on a plasmid) or on a separate fragment. Following transfection transformants are screened for the presence of this selection marker gene and subsequently analyzed for the presence of the gene(s) of interest. A selectable marker is a product, which provides resistance against a biocide or virus, resistance to heavy metals, prototrophy to auxotrophs and the like. Useful selectable markers include amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC or sutB (sulfate adenyltransferase), trpC (anthranilate synthase), ble (phleomycin resistance protein), or equivalents thereof.

The obtained host cell may be used for producing pravastatin.

In a third aspect, the present invention provides a method for isolating polynucleotides encoding polypeptides capable of improving the compactin into pravastatin conversion of the second aspect, comprising the steps of:
(i) transforming a host cell with a polynucleotide of the first aspect of the invention;
(ii) selecting clones of transformed cells for their capacity to hydroxylate compactin;
(iii) re-transforming these isolated clones with various polynucleotides;
(iv) selecting clones of transformed cells for their improved capacity to hydroxylate compactin;
(v) isolating the plasmids;
(vi) sequencing the inserts of the plasmids

The various polynucleotides of step (iii) can be obtained from several sources. It can be genomic DNA, copy DNA, RNA, semi-synthetic or from synthetic origin. It can be from a eukaryotic or a prokaryotic host. It can be provided as circular or linear polynucleotides. It can be specific polynucleotides (i.e. a gene or a gene family or an error prone library derived from a gene) or it can be random polynucleotides (i.e. a metagenomic library or random digested genomic DNA). It can be expressed from its own promoter or it can be cloned behind a promoter functional in the host of step (i).

Such a nucleic acid sequence encoding a polypeptide that improves compactin hydroxylase activity may also be isolated by e.g. screening a genomic or cDNA library of the donor microorganism of the polynucleotide of the first aspect. Once a nucleic acid sequence homologous to a probe derived from SEQ ID NO 2 is detected, the sequence and its surrounding DNA may be isolated or cloned by utilizing techniques which are known to those of ordinary skill in the art (see Sambrook et al., 1989, Molecular Cloning, A Laboratory Manual, 2d edition, Cold Spring Harbor, N.Y.).

In this way one is enabled to clone variant polynucleotides that encode polypeptides with enhanced function, or polynucleotides that encode polypeptides that assist or facilitate the functioning of the compactin hydroxylase enzyme, or polynucleotides that activate the promoter in front of the compactin hydroxylase gene.

In one embodiment, disclosed is a method to improve the efficiency of the compactin into pravastatin conversion by isolating a redox regenerating system, which is needed for a p450 enzyme (Pylypenko and Schlichting, 2004, Annu. Rev. Biochem. 73:991-1018) and introducing this in the compactin hydroxylase expressing host cell. The general methods of introducing such as system in the host cell are the same as described for introducing the compactin hydroxylase and outlined above. Such redox regenerating system may be obtained from species cited as species from which the polynucleotide of the second aspect may be obtained or heterologously expressed in; examples of which are, but are not limited to, Streptomyces species (i.e. Streptomyces carbophilus, Streptomyces flavidovirens, Streptomyces coelicolor, Streptomyces lividans, Streptomyces exfoliates, Streptomyces avermitilis, Streptomyces clavuligerus) or Amycolatopsis species (i.e. Amycolatopsis orientalis) or Bacillus species (i.e. Bacillus subtilus, Bacillus amyloliquefaciens, Bacillus licheniformis) or Corynebacterium species (i.e. Corynebacterium glutamicum) or Escherichia species (i.e. Escherichia CA. Also alternative systems can be applied. Examples of alternative systems are, but not limited to, integrating the compactin hydroxylase of the present invention in a class IV p450 system, thereby fusing it to the redox partners (Roberts et al., 2002, J. Bacteriol. 184:3898-3908 and Kubota et al., 2005, Biosci. Biotechnol. Biochem. 69:2421-2430) or by NAD(P)H generating non-p450 linked enzymes like phosphite dehydrogenase (Johannes et al., 2005, Appl Environ Microbiol. 71:5728-5734.) or by non-enzymatic means (Hollmann et al., 2006, Trends Biotechnol. 24:163-171).

In a fourth aspect of the present invention, the pravastatin produced according to the method of the third aspect is comprised within a pharmaceutical composition.

### LEGENDS TO THE FIGURES

FIG. 1 shows the conversion catalyzed by the product of the cmpH gene, compactin hydroxylase. Legend: [C]=compactin; [P]=pravastatin.

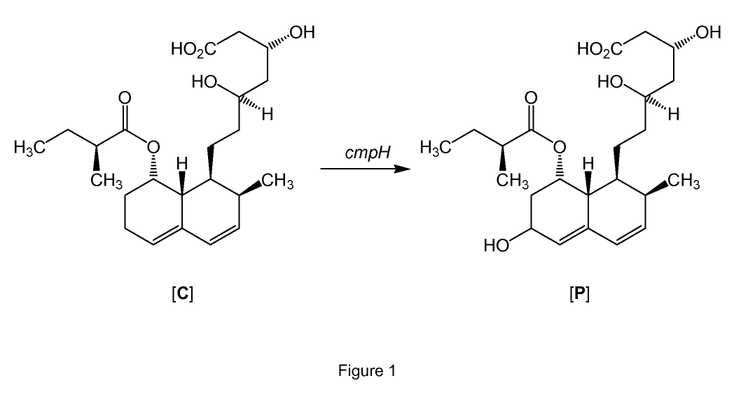

FIG. 2 shows the plasmid pZERO-Ao-11H9. Legend: ORF-1=first Open Reading Frame, ORF-2=second Open Reading Frame, zeo=gene encoding resistance to zeocin, kan=gene encoding resistance to kanamycin.

FIG. 3 shows the plasmid pZERO-Ao-11H9d. Legend: ORF-1=first Open Reading Frame, zeo=gene encoding resistance to zeocin, kan=gene encoding resistance to kanamycin.

FIG. 4 shows the plasmid pACYC-taqScp450. Legend: Sc-p450=Streptomyces carbophilus gene encoding compactin hydroxylase p450, cat=gene encoding resistance to chloramphenicol.

FIG. 5 shows the plasmid pACYC-taqAop450. Legend: A0-cmpH=Amycolatopsis orientalis gene encoding compactin hydroxylase p450, cat=gene encoding resistance to chloramphenicol.

### EXAMPLES

### General Methods

Standard DNA procedures and prokaryotic cultivations were carried out as described elsewhere (Sambrook, J. et al., 1989, Molecular cloning: a laboratory manual, 2^{nd} Ed., Cold Spring Harbor Laboratory Press, Cold Spring Harbor, N.Y.). DNA was amplified using the proofreading enzyme Phusion polymerase (Finnzymes). Restriction enzymes were from Invitrogen or New England Biolabs. Hydrolysis of compactin was done by dissolving compactin in ethanol to a final concentration of 20 mg/ml. NaOH was added from a 4M stock, to a final concentration of 0.1M. The solution was heated at 50° C. for 1 to 2 hrs and subsequently cooled to rT. This solution can be stored at rT for 3 months. Pravastatin and non-hydrolyzed compactin stock solutions were made by dissolving both compounds in ethanol at 20 mg/ml.

### Example 1

### Screening for Efficient Whole-Cell Compactin to Pravastatin Bioconversion

Diverse prokaryotic and fungal species (Table 1) were tested to isolate a species with improved conversion from hydrolyzed compactin. All species were pre-cultured for 1-3 days (depending on the growth rate of the species) in 25 ml 2×YT medium, washed and suspended in 25 ml fresh 2×YT medium. After an adaptation period of several hours while shaking at 280 rpm and 30° C., hydrolyzed compactin was added at final concentrations of 0.1, 0.2, 0.5 and 1 mg/ml. After 24 h incubation the broths were collected by transferring the content of the shake flasks into a 50 ml Greiner tube.

Samples were frozen at −20° C., followed by freeze drying. The statins were extracted by addition of 1-2 ml methanol to the freeze-dried samples, followed by repeated vortexing. The solids were separated from the liquid phase by centrifugation. 200 μl of the methanol-extract was transferred into an HPLC vial followed by HPLC analysis as follows:

| Eluens: | A: 33% acetonitrile, 0.025% trifluoroacetic | | |
|---|---|---|---|
| | acid in milliQ water | | |
| | B: 80% acetonitrile in MilliQ water | | |
| Gradient: | Time (min) | % eluent A | % eluent B |
| | 0-8 | 100 | 0 |
| | 8-8.1 | 100→0 | 0→100 |
| | 8.1-12 | 0 | 100 |
| | 12-13 | 0→100 | 100→0 |
| | 13-14 | 100 | 0 |

| | |
|---|---|
| Column: | Waters XTerra RP18 |
| | (Column Temp. = Room temperature) |
| Flow: | 1 ml/min |
| Injection vol.: | 10 μl; (Tray Temp. = Room temperature) |
| Instrument: | Waters Alliance 2695 |
| Detector: | Waters 996 Photo Diode Array |
| Wavelength: | 238 nm |
| Retention time: | Pravastatin 4 min, hydr. compactin 10.4 min, |
| | compactin 10.9 min |

TABLE 1
Prokaryotic species tested for hydroxylation
of hydrolyzed compactin

| | | Hydrolyzed |
|---|---|---|
| Species | Strain | compactin→pravastatin |
| Escherichia coli | DH10b | − |
| Choanephora circinans | CBS153.58 | − |
| Helicostylum piriforme | VKM-F1068 | − |
| Rhizopus microsporus | VKM-F1218 | − |
| chinensis | | |
| Rhizopus stolonifer stolonifer | CBS 382.52 | − |
| Actinokineospora riparia | VKM-Ac1980 | +/− |
| Pseudonocardia alni | VKM-Ac916 | +/− |
| Streptomyces carbophilus | FERM-BP1145 | ++ |
| Amycolatopsis orientalis | NRRL18098 | ++++ |

As can be seen in Table 1 pravastatin was synthesized by four species of the tested set: Actinokineospora riparia, Pseudonocardia alni, Streptomyces carbophilus and Amycolatopsis orientalis.

### Example 2

### Biological Hydrolysis of Compactin

To establish if the species described in Example 1 could also hydrolyze and/or hydroxylate compactin in lactone-form, four selected species were pre-cultured for 1-3 days (depending on the growth rate of the species) in 25 ml 2×YT medium, washed and resuspended in 25 ml fresh 2×YT medium. After adaptation for several hours while shaking at 280 rpm and 30° C., non-hydrolyzed compactin was added at 0.2 mg/ml. After 24 h incubation the broths were collected by transferring the content of the shake flasks into a 50 ml Greiner tube. Samples were frozen at −20° C., followed by freeze drying. The statins were extracted by addition of 1-2 ml methanol, followed by repeated vortexing. The solids were separated from the liquid phase by centrifugation. 200 μl of the methanol-extract was transferred into an HPLC vial followed by HPLC analysis as described in Example 1. All four species (Actinokineospora riparia, Escherichia coli, Streptomyces carbophilus and Amycolatopsis orientalis) hydrolyze compactin, but this is not a prerequisite for pravastatin formation. Amycolatopsis orientalis was the most efficient species in synthesizing pravastatin.

TABLE 2
Prokaryotic species tested for hydrolysis and/or hydroxylation of compactin

| | Compactin | | Hydrolyzed | | Unknown |
|---|---|---|---|---|---|
| | added | Compactin | compactin | Pravastatin | product |
| Amycolatopsis orientalis | 0.2 mg/ml | 4% | 1% | 16% | 81% |
| Actinokineospora riparia | 0.2 mg/ml | 75% | 25% | <1% | |
| Escherichia coli | 0.2 mg/ml | 49% | 51% | | |
| Streptomyces carbophilus | 0.2 mg/ml | 14% | 78% | 8% | |

### Example 3

### Amycolatopsis Orientalis has Very Efficient Compactin Hydroxylation

From Example 1 it was concluded that Amycolatopsis orientalis was superior for compactin hydroxylation to Streptomyces carbophilus. For further study both species were pre-cultured for 24 h in 25 ml 2×YT medium, washed and resuspended in 25 ml fresh 2×YT medium. After several hours while shaking at 280 rpm and 30° C., hydrolyzed compactin was added at 0.1 and 0.2 mg/ml. After 24 h incubation the broths were collected by transferring the content of the shake flasks into a 50 ml Greiner tube. Samples were frozen at −20° C., followed by freeze drying. The statins were extracted by addition of 1-2 ml methanol to the dried samples, followed by repeated vortexing. The solids were separated from the liquid phase by centrifugation. 200 μl of the methanol-extract was transferred into an HPLC vial followed by HPLC analysis as described in Example 1. As can be seen from Table 3, Amycolatopsis orientalis is capable of converting compactin into pravastatin with 100% efficiency where Streptomyces carbophilus is not.

TABLE 3
Comparison in compactin hydroxylation by Amycolatopsis orientalis
and Streptomyces carbophilus.

| | Hydrolyzed | | | |
|---|---|---|---|---|
| | compactin | Hydrolyzed | | Unknown |
| | added (mg/ml) | compactin | Pravastatin | Product |
| Amycolatopsis | 0.1 | | 100% | |
| orientalis | 0.2 | 6% | 88% | 6% |
| Streptomyces | 0.1 | 14% | 86% | |
| carbophilus | 0.2 | 16% | 84% | |

### Example 4

### Isolating Gene Fragment Encoding the Biocatalyst Converting Compactin to Pravastatin

Gene Library Amycolatopsis orientalis

A colony of Amycolatopsis orientalis in liquid medium (10 g/l glucose, 5 g/l yeast extract, 20 g/l starch, 1 g/l CaCO₃ and 0.5 g/l casaminoacids, baffled flasks) was grown at 28° C. until OD=2.0. Part was used to prepare glycerol stocks and part was used to inoculate (ratio 1/50) a flask with 50 ml of the liquid medium for the preparation of cells for genomic DNA isolation. After 16 h at 28° C. the culture was used for isolating genomic DNA. Thereto, during the last hour of the incubation ampicillin was added to a final concentration of 200 μg/ml. Cells were harvested by centrifugation (15 min at 8000 rpm) and the pellet was resuspended in 5 ml of 50 mM Tris-HCl with 50 mM EDTA adjusted to pH 8.0. After adding 100 μl of lysozyme (100 mg/ml) and 40 μl proteinase K (20 mg/ml), the suspension was incubated for 30 min at 37° C. Nuclei Lysis Solution (6 ml) of Promega was added. Incubation for 15 min at 80° C. and 30 min at 65° C. led to almost complete cell lysis. After RNase treatment (10 μl of 100 mg/ml RNaseA solution), 2 ml of Protein Precipitation Solution of Promega was added, the mixture was vortexed (20 s) and incubated on ice (15 min). After centrifugation (15 min at 5000 rpm) the supernatant was mixed with 0.1 volumes of NaAc (3 M, pH 5) and 2 volumes of EtOH (96%). The visible complexes of precipitated genomic DNA were transferred with a Pasteur pipette and dissolved in 500 μl of 10 mM Tris (pH 8.0). A second proteinase K treatment (10 μl of the 20 mg/ml stock solution was used per 200 μl sample, followed by 30 min incubation at 37° C.) was applied to remove remaining proteins. After the proteinase K step, 500 μl of phenol/chloroform/isoamylalcohol (PCI, 25:24:1) was added and the mixture was centrifuged for 5 min at 14,000 rpm. The upper phase was transferred to a new vial and 500 μl of PCI (24:1) was added for removing traces of phenol. The phases were separated by centrifugation and the upper phase was mixed with 0.1 volumes of NaAc (3 M, pH 5) and 2 volumes of EtOH (96%) for precipitation of the DNA. The genomic DNA was taken out with a pipette, washed with 70% cold EtOH and dissolved in 500 μl of Tris-EDTA buffer. This resulted in 134 μg of purified genomic DNA with an A260 nm/A280 nm of 1.85. Sau3Al (0.067 units/μg DNA) was used to partially digest isolated Amycolatopsis orientalis DNA to obtain smaller fragments ranging from 4 to 10 kb. Genomic DNA (50 μg) was digested and the fragments between 4 and 10 kb were isolated from a preparative 0.6% agarose gel using the Qiagen QIAquick extraction kit and finally dissolved in 20 μl 10 mM Tris, pH 8.0. These fragments were ligated to BamHI digested pZErO-2 (Invitrogen) and transformed into Escherichia coli DH10B, resulting in about 39,000 colonies. Twenty individual colonies were used to inoculate 10 ml 2×YT medium to check the diversity of the library and to determine the average insert size. 19 plasmids contained inserts of different sizes, whereas one colony had no insert (self-ligated vector 5%). The average insert size of the gDNA fragments in pZErO-2 was about 3.8 kb. All obtained transformants were collected from the plates and resuspended in liquid 2×YT medium with kanamycin and glycerol was added to a final concentration of 8% (v/v) and stored at −80° C.

### Screening the Amycolatopsis Orientalis Gene Library

The Amycolatopsis orientalis gene library was plated on 2×YT agar+kanamycin (50 mg/L) and incubated for 72 h at rT. Almost 12,000 colonies were used to inoculate 120 96-wells microtiterplates (MTPs), containing 0.2 ml 2×YT medium+35 mg/L kanamycin. The MTPs were incubated at 25° C. with 500 rpm for 48 h. From each well 140 μl cell suspension was centrifuged for 10 min at 3,000 rpm and the supernatant was discarded by tapping the plates on tissue paper (the remainder of the culture was added to 50 μl 20% glycerol and stored at −80° C.). Per well 250 μl of substrate solution (2×YT medium with hydrolyzed compactin, 200 mg/L; glucose, 2 g/l; phosphate buffer, 50 mM; pH 6.8). Cell pellets in the wells were resuspended and incubated for 48 h at 30° C., 280 rpm. The statins were extracted after addition of 0.35 ml methanol per well and one hour mixing at 280 rpm. Cell debris was removed by centrifugation for 15 minutes at 2750 rpm. Samples of 100 μl were analyzed by LC-MS.

LC-MS Analysis of the Amycolatopsis orientalis Gene Library

The compactin standard was prepared by hydrolysis of mevastatin (A.G. Scientific, lot no. A7413, purity 99.36%) with 1.5 M NaOH in MeOH (1:2) during the night under stirring in helium atmosphere 2. The pH is reduced by addition of HCl (4 M) and the standard is further diluted with water. The samples are analyzed on a Waters LC/MS system with a short (20 mm) CN column of ACT (Advanced Chromatography Technologies) with water and acetonitrile (both with 0.1% formic acid) as mobile phases. The details of the LC-part are:

| Apparatus: | Waters Alliance 2795 LC | | | | |
|---|---|---|---|---|---|
| Mobile phases: | Solvent A: | Water with 0.1% formic acid | | | |
| | Solvent B: | Acetonitrile with 0.1% formic acid | | | |
| Needle wash: | 50% MilliQ water + 50% acetonitrile | | | | |
| Gradient timetable: | Time (min) | A % | B % | flow (ml/min) | curve |
| | 0.00 | 80.0 | 20.0 | 1.00 | 1 |
| | 0.35 | 80.0 | 20.0 | 1.00 | 6 |
| | 1.00 | 20.0 | 80.0 | 1.00 | 6 |
| | 1.40 | 20.0 | 80.0 | 1.00 | 6 |
| | 1.50 | 80.0 | 20.0 | 1.00 | 6 |
| | 2.00 | end | | | |

| | |
|---|---|
| Column: | ACT, ACE 3 CN, 20 × 2.1 mm, particle size 3 μm |
| Column temp.: | 25° C. |
| Injection volume: | 5 μl |

In the MS electrospray ionization in the positive mode (ES+) is used and the compounds are analyzed as the sodium adducts ([M+Na]+) with selected ion (SIR) of the three compounds. The details of the MS-part are:

| Apparatus: | Waters ZQ 2000 | | Source: | ES+ | |
|---|---|---|---|---|---|
| Capillary: | 3.50 | kV | Cone: | 30 | V |
| Desolation temp.: | 360° | C. | Source temp.: | 140° | C. |
| Extractor: | 2 | V | RF lens: | 0.3 | V |
| Cone Gas flow: | 130 | l/hour | Desolvation flow: | 610 | l/hour |
| LM 1 Resolution: | 15.0 | | HM 2 Resolution: | 15.0 | |
| Energy 1: | 0.1 | | Multiplier: | 650 | V |

Scan mass range (m/z) 200-600 amu, scan duration 0.20 s, interscan delay 0.05 s; SIR of 3 channels: 413.30, 431.3, 447.3, dwell 0.07 s, interscan delay 0.05 s.

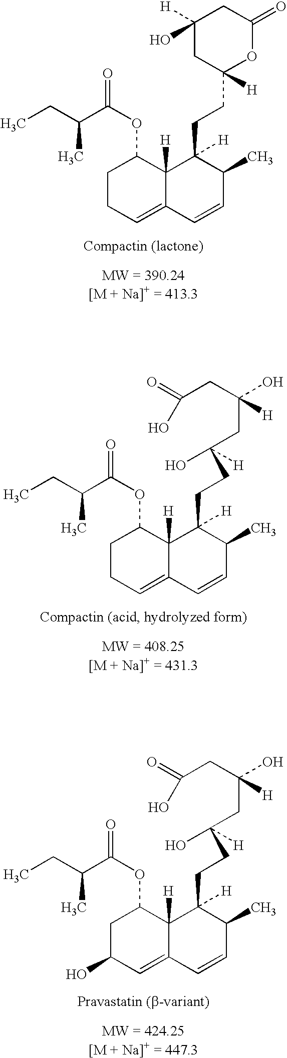

With this set-up it is possible to distinguish the four most important molecules, compactin, hydrolyzed compactin and the two stereoisomers of 6-hydroxy-compactin, i.e. the β-variant pravastatin (structure see above) and the α-variant epi-pravastatin.

TABLE 4
Example of results from MTP screening for compactin hydroxylase

| | Well | Area | Area |
|---|---|---|---|
| Nr. | position | pravastatin | compactin |
| 1 | B.1 | 18076 | 3664332 |
| 2 | B.3 | <10000 | 3409885 |
| 3 | B.5 | 10338 | 3176999 |
| 4 | B.7 | <10000 | 3421702 |
| 5 | B.9 | <10000 | 3719904 |
| 6 | B.11 | <10000 | 3202990 |
| 7 | B.13 | <10000 | 3237775 |
| 8 | B.15 | <10000 | 3473592 |
| 9 | B.17 | <10000 | 3638848 |
| 10 | B.19 | <10000 | 3694601 |
| 11 | B.21 | <10000 | 3606196 |
| 12 | B.23 | <10000 | 3710229 |
| 13 | D.1 | <10000 | 3615275 |
| 14 | D.3 | <10000 | 3396888 |
| 15 | D.5 | <10000 | 3487926 |
| 16 | D.7 | <10000 | 3443088 |
| 17 | D.9 | <10000 | 3483672 |
| 18 | D.11 | <10000 | 3683947 |
| 19 | D.13 | <10000 | 3565824 |
| 20 | D.15 | <10000 | 3610494 |
| 21 | D.17 | <10000 | 3719262 |
| 22 | D.19 | <10000 | 3856010 |

Several clones were identified as candidates as they showed a small, but significant peak at the position of pravastatin. In Table 4 as example a subset of the results is shown in which one clone gives a signal above background (clone in well position B1).

### Example 5

### Identification of Gene Encoding Compactin to Pravastatin Biocatalyst

Retesting the Putative Compactin Hydroxylases from Amycolatopsis orientalis

The analysis of Example 4 was repeated with four clones which were identified in the first round as putative clones. However, now the clones were grown in shake flasks in stead of MTPs and some cultivation conditions were varied. The clones were pre-cultivated in 10 ml 2×YT which was inoculated with the Escherichia coli cells containing putative compactin hydroxylases and grown for 24 hours at 30° C., 280 rpm. Subsequently, 0.1-0.5 mM IPTG and 0.5 mM β-aminolevulinate was added and the cultures were incubated at 22° C., 280 rpm for 12 h. Cells were harvested, washed and resuspended by vortexing in fresh 2×YT medium (supplemented with hydrolyzed compactin, 200 mg/L; glucose, 2 g/l; phosphate buffer, 50 mM; pH 6.8). Cell suspensions were incubated either at 30 or 37° C., for 24 or 48 hours, at 280 rpm. The statins were extracted and analyzed as described in Example 4.

TABLE 5
Results of retesting putative compactin
hydroxylases from A. orientalis.

| Clone | Condition | Pravastatin (peak area) |
|---|---|---|
| 11H9 | 24 hr, 37 C. | 39964580 |
| 12H9 | 24 hr, 37 C. | 1914055 |
| 15H2 | 24 hr, 37 C. | 1951158 |
| 16H2 | 24 hr, 37 C. | 2120422 |
| control | | 1894831 |
| 11H9 | 48 hr, 30 C. | 26749341 |
| 12H9 | 48 hr, 30 C. | 2542003 |
| 15H2 | 48 hr, 30 C. | 2388226 |
| 16H2 | 48 hr, 30 C. | 1565684 |
| 11H9 | 48 hr, 37 C. | 48316813 |
| 12H9 | 48 hr, 37 C. | 2613137 |
| 15H2 | 48 hr, 37 C. | 2408859 |
| 16H2 | 48 hr, 37 C. | 2934999 |

As can be seen in Table 5 only clone 11H9 has a real significant conversion of compactin to pravastatin. This one was therefore selected for further analyses.

### Sequencing and Sequence Analysis

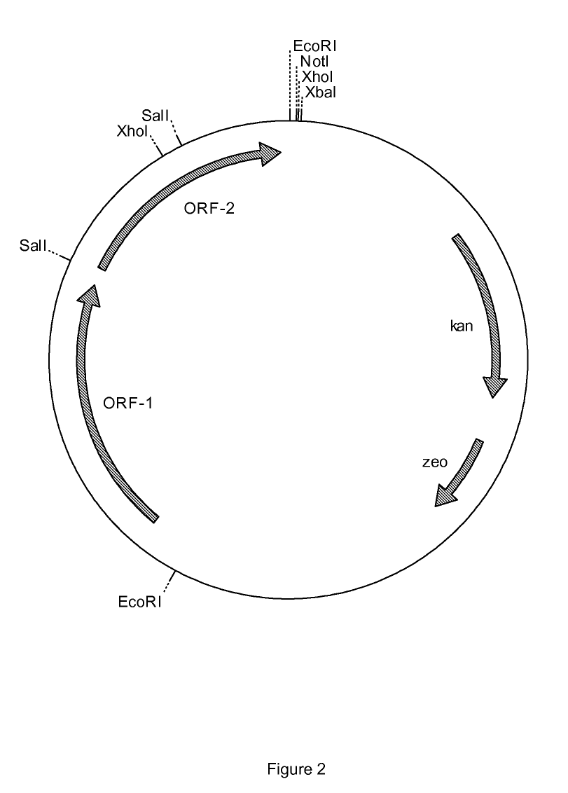

Escherichia coli clone 11H9 was cultivated in 2×YT with kanamycin and plasmid DNA was isolated using the Qiagen QIAprepep kit and the sequence of the Amycolatopsis orientalis genome insert in the pZERO-2 plasmid was determined. The sequence of the insert is 2545 nucleotides long (see SEQ ID NO. 1). Analyses of the DNA sequence were performed and two Open Reading Frames (ORFS) could be identified (FIG. 2). The first ORF encodes a putative protein of 401 amino acids (SEQ ID no. 2 and 3) which has some homology to known p450 enzymes (i.e. the best being Cytochrome p450 monooxygenase CYP105S2 from Streptomyces tubercidicus). The second ORF has some membrane spanning regions and might encode an ATP-type Binding Cassette (ABC) protein.

Identification of the Structural Gene, cmpH

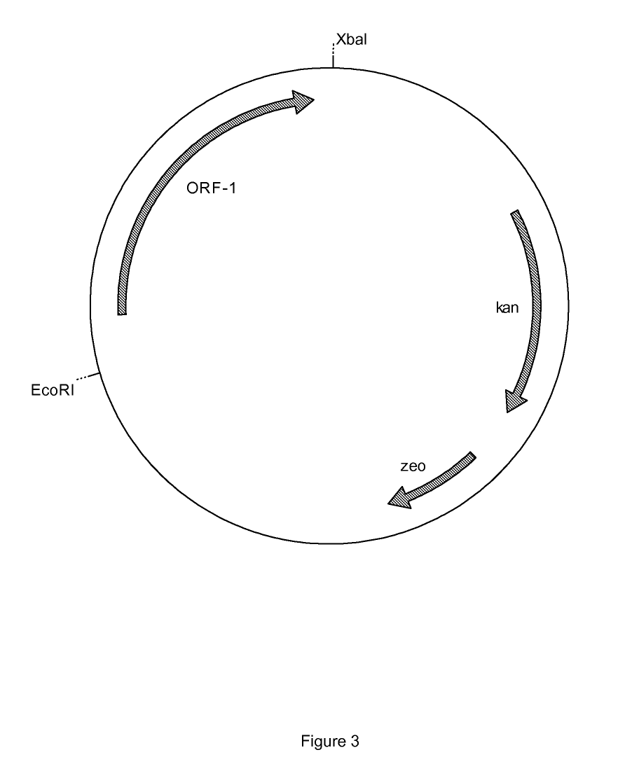

To identify the structural gene capable of hydroxylating compactin, ORF-2 was deleted from pZERO-Ao-11H9 via double digestion with SalI and XhoI. Subsequently, the 4.9 kb fragment was isolated and self-ligated. The resulting plasmid pZERO-Ao-11H9 contains only ORF-1 as a full ORF (FIG. 3). This clone has the same conversion rate as clone pZERO-Ao-11H9, indicating that ORF-1 encodes a functional compactin hydroxylase, named cmpH.

### Comparative Example 6

Activity of Streptomyces carbophilus Compactin Hydroxylase in Escherichia coli

Construction of p450-SCA E. coli Expression Clone

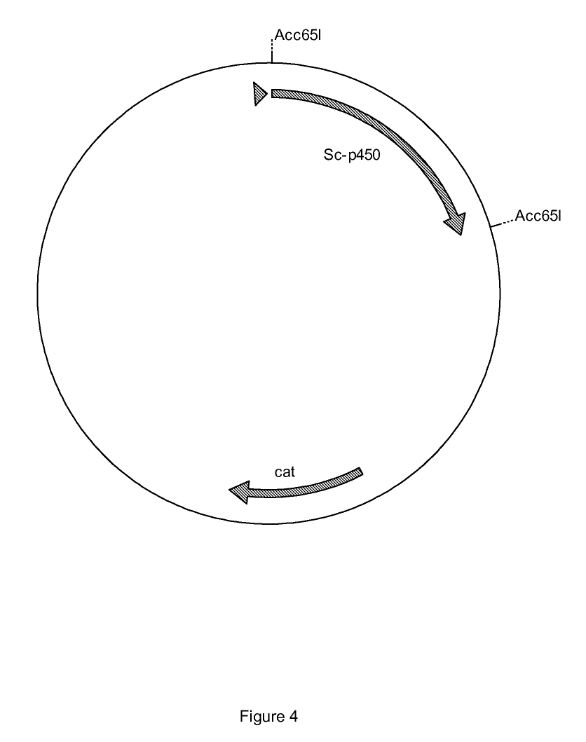

The gene encoding the Streptomyces carbophilus p450 was PCR amplified from genomic DNA isolated from strain FERM-BP1145 using the primers of SEQ ID NO. 7 and SEQ ID NO. 8. The PCR fragment was clone in the pCR2.1TOPO/TA vector according to the supplier's instructions (Invitrogen). The expression clone was constructed by digesting pACYC-taq (Krämer, M., 2000. Untersuchungen zum Einfluss erhöhter Bereitstellung von Erythrose-4-Phosphat and Phosphoenolpyruvat auf den Kohlenstoffluss in den Aromatenbiosyntheseweg von Escherichia coli. Berichte des Forschungszentrums Jülich 3824, ISSN 0944-2952, PhD Thesis, University of Düsseldorf) with Acc651 and ligating the Acc651 fragment isolated from the pCR2.1TOPO/TA vector, resulting in pACYC-taqScp450 (FIG. 4).

Activity Determination in Escherichia coli Extracts

Escherichia coli cells containing pACYC-taqScp450 were cultivated in 10 ml 2×YT with chloramphenicol. Cell suspensions were basically processed and incubated with compactin as described in example 5. In this case cultivation temperature was 37 degrees Celsius and chloramphenicol and IPTG (0.1 mM) were added to the reaction mixture. The reactions were incubated at 30° C. and 220 rpm. Samples were taken at various time points and analyzed using the HPLC protocol as described in Example 1. After 24 h no pravastatin could be determined.

### Example 7

### Activity of Amycolatopsis orientalis Compactin Hydroxylase in Escherichia coli

Construction of Ao-cmpH Escherichia coli Expression Clone

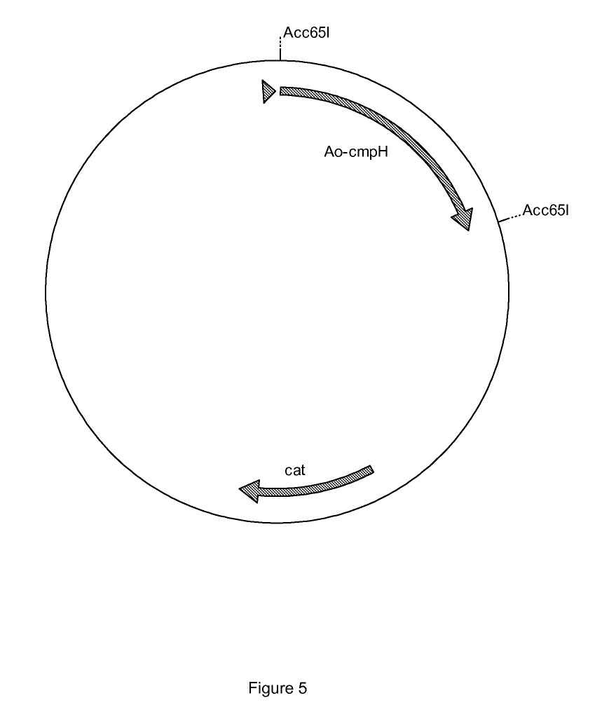

The gene encoding the Amycolatopsis orientalis p450 was PCR amplified from genomic DNA isolated using the primers of SEQ ID NO 9 and SEQ ID NO 10. The PCR fragment was cloned in the pCR2.1TOPO/TA vector according to the supplier's instructions (Invitrogen). The expression clone was constructed by digesting pACYC-taq (Kramer, 2000) with Acc65l and ligating the Acc65l fragment isolated from the pCR2.1TOPO/TA vector, resulting in pACYC-taqAop450 (FIG. 5).

Activity Determination in Escherichia coli Extracts

Escherichia coli cells containing pACYC-tagAop450 (FIG. 5) were cultivated in 2×YT (10 ml) with chloramphenicol. Cell suspensions were incubated with compactin as described in Example 5. Cultivation temperature was 37° C. and chloramphenicol and IPTG (0.1 mM) were added. The reactions were incubated at 30° C. and 220 rpm. Samples were taken at various time points and analyzed using the HPLC protocol as described in Example 1. After 24 h a large peak of pravastatin was detected. This result clearly demonstrates that the Amycolatopsis orientalis p450 enzyme is much better suitable for hydroxylating compactin in Escherichia coli than other p450's.

TABLE 6
Compactin conversion of Escherichia coli
strains harboring the P450 genes.

| | Pravastatin β-variant | Pravastatin α-variant |
|---|---|---|
| Strain tested | Ratio in % | Ratio in % |
| E. coli Top10 (Invitrogen) | 5-10% | 90-95% |
| pACYC-taqAop450 | | |

The data summarize average conversion ratios of 50 tested clones, each giving a compactin to pravastatin conversion of at least 90%. Percentages refer to converted compactin.

### Example 8

### Activity of the Derivatives of the Amycolatopsis orientalis Compactin Hydroxylase in Escherichia coli

The genes SEQ ID NO 11-18, 27-34 were produced synthetically and used as template for PCR reactions with oligonucleotides SEQ ID NO 9 and 10 with added attB1 (for SEQ ID NO 9) and attB2 (for SEQ ID NO 10) recombination sites. The PCR fragments were cloned in the pDONR221 vector (Invitrogen Corporation, The Netherlands) by performing the Gateway BP reaction (Invitrogen Corporation); the sequences were verified by DNA sequencing to exclude PCR related errors. Using the Gateway LR reaction, the genes were transferred from the pDONR221 vector to the pET-DEST42 vector, resulting in the final expression vectors pET-DEST42-P450. Escherichia coli BL21 DE3 harboring pET-DEST42-P450 (SEQ ID NO 11-18, 27-34 as inserts) was cultivated in 10 ml 2×YT with kanamycin at 30° C. until OD₆₀₀=0.5-1.0 Subsequently the culture was supplemented with 0.1-0.3 mM IPTG and 0.5 mM β-aminolevulinate and incubated at 22° C. at 280 rpm for 12 h. Cells were harvested, washed and resuspended in fresh 2×YT medium (supplemented with hydrolyzed compactin, 200 mg/L; glucose, 2 g/l; phosphate buffer, 50 mM; pH 6.8). Cell suspensions were incubated either at 30 or 37° C., for 24 or 48 h, at 280 rpm. The statins were extracted and analyzed as described in Example 4. After 24 h, a very large pravastatin peak could be identified. In contrast to the experiment described in example 7, the majority of produced pravastatin is the β-variant, giving proof for the significantly changed stereospecificity of the enzymes of SEQ ID NO 19-26 and SEQ ID NO 35-42, respectively, if compared to the compactin hydroxylase enzyme encoded by SEQ ID NO 3 or SEQ ID NO 6.

TABLE 7
Compactin conversion of Escherichia coli
strains harboring the P450 genes.

| | Pravastatin β-variant | Pravastatin α-variant |
|---|---|---|
| Strain tested | Ratio in % | Ratio in % |
| E. coli BL21 DE3 | 90-95% | 5-10% |
| (Invitrogen) | | |
| pET-DEST42-P450 | | |
| (SEQ ID NO 11-18, 27-34) | | |

The data summarize average conversion ratios of 50 tested clones each giving a compactin to pravastatin conversion of at least 90%. All DNA fragments encoded by the SEQ ID NO 11-18, 27-34 catalyze very similar compactin conversion features as shown below. Percentages refer to converted compactin.

1. A polypeptide selected from the group consisting of a polypeptide having an amino acid sequence according to SEQ ID NO 3, SEQ ID NO 6, SEQ ID NO 43-59, a polypeptide having an amino acid sequence that has a degree of identity to SEQ ID NO 3 of at least 50%, a polypeptide having an amino acid sequence that has a degree of identity to SEQ ID NO 6 of at least 60% and a polypeptide having an amino acid sequence differing no more than 3 amino acids from SEQ ID NO 43-59.

2. Polypeptide according to claim 1 having an amino acid sequence according to SEQ ID NO 3, SEQ ID NO 6, SEQ ID NO 19-26 or SEQ ID NO 35-59 or having an amino acid sequence that has a degree of identity to SEQ ID NO 3, SEQ ID NO 6, SEQ ID NO 19-26 or SEQ ID NO 35-59 of at least 90%.

3. A polypeptide capable of converting compactin in to pravastatin with an efficiency of at least 50%.

4. A polynucleotide comprising a DNA sequence encoding the polypeptide of claim 1.

5. The polynucleotide of claim 4 that is SEQ ID NO 1, 2, 4 or 5.

6. A method for producing pravastatin comprising:
(i) expressing a polynucleotide of claim 4 in a production host;
(ii) growing the production host obtained in (i); and
(iii) isolating pravastatin from the mixture obtained in (ii).

7. A method for isolating polynucleotides encoding polypeptides capable of improving the compactin into pravastatin conversion, comprising:
(i) transforming a host cell with a polynucleotide of claim 4;
(ii) selecting clones of transformed cells for their capacity to hydroxylate compactin;
(iii) re-transforming these isolated clones with various polynucleotides;
(iv) selecting clones of transformed cells for their improved capacity to hydroxylate compactin;
(v) isolating the plasmids; and
(vi) sequencing the inserts of the plasmids.

8. A method for producing pravastatin comprising:
(i) co-expressing a polynucleotide comprising a DNA sequence encoding a polypeptide selected from the group consisting of a polypeptide having an amino acid sequence according to SEQ ID NO 3, SEQ ID NO 6, SEQ ID NO 43-59, a polypeptide having an amino acid sequence that has a degree of identity to SEQ ID NO 3 of at least 50%, a polypeptide having an amino acid sequence that has a degree of identity to SEQ ID NO 6 of at least 60% and a polypeptide having an amino acid sequence differing no more than 3 amino acids from SEQ ID NO 43-59 and a polynucleotide according to claim 7 in a production host;
(ii) growing the production host obtained in (i); and
(iii) isolating pravastatin from the mixture obtained in (ii).

9. Method according to claim 8 wherein compactin is added during growth of said production host.

10. Method according to claim 8 wherein said production host is a fungal cell or a bacterial cell.

11. Method according to claim 10 wherein said fungal cell is yeast or a filamentous fungal cell and said bacterial cell is selected from the group consisting of Actinomycetes and Proteobacteria.

12. Method according to claim 11 wherein said yeast is Saccharomyces cerevisiae, Hansenula polymorpha, Kluyveromyces lactis or Pichia pastoris and said filamentous fungal cell is Aspergillus terreus, Aspergillus nidulans, Aspergillus niger, Penicillium citrinum, Penicillium chrysogenum, Monascus ruber or Monascus paxii and said Actinomycetes is Streptomyces, Amycolatopsis or Actinomadura and said Proteobacteria is Escherichia or Bacillus.

13. Method according to claim 12 wherein said Streptomyces is Streptomyces carbophilus, Streptomyces lividans, Streptomyces coelicolor or Streptomyces clavuligerus and said Amycolatopsis is Amycolatopsis orientalis and said Escherichia is Escherichia coli and said Bacillus is Bacillus amyloliquefaciens, Bacillus licheniformis or Bacillus subtilis.

14. A pharmaceutical composition comprising pravastatin obtained according to claim 6.
